(19) United States
(12) Patent Application Publication
(10) Pub. No.: US 2020/0390988 A1
(43) Pub. Date: Dec. 17, 2020

(54) SPACER DEVICE FOR AN INHALER AND METHOD OF MANUFACTURE THEREOF

(71) Applicant: NATIONAL UNIVERSITY OF SINGAPORE, Singapore (SG)

(72) Inventors: Shi Yun Karyn LIM, Singapore (SG); John ZHENG, Singapore (SG); Kum Yoong Marcus WOO, Singapore (SG); Wee Chuan Melvin LOH, Singapore (SG); Ching Chiuan YEN, Singapore (SG); Felix AUSTIN, Singapore (SG); Ulrich Bohannes SCHRAUDOLPH, Singapore (SG); Hugo VAN BEVER, Singapore (SG)

(73) Assignee: NATIONAL UNIVERSITY OF SINGAPORE, Singapore (SG)

(21) Appl. No.: 16/954,899

(22) PCT Filed: Dec. 14, 2018
(86) PCT No.: PCT/SG2018/050613

§ 371 Date: Jun. 17, 2020

(30) Foreign Application Priority Data
Dec. 18, 2017 (SG) 10201710518Y

(51) Int. Cl. A61M 15/00; A61M 39/10; A61M 16/06

(52) U.S. Cl. CPC A61M 15/009; A61M 15/0018; A61M 39/10; A61M 15/0021; A61M 2207/10; A61M 16/06; A61M 2205/583; A61M 2205/073; A61M 15/0088

(57) ABSTRACT

A spacer device for an inhaler. The device may include a hollow tube. The device may also include a piston member configured to be inserted into the hollow tube such that a space between a first end of the hollow tube and the piston member may define a chamber for holding a mist released from the inhaler. The piston may be slidable along the hollow tube towards the first end of the hollow tube as the mist is aspirated from the chamber. The hollow tube and the piston member may cooperate to provide a visual indication of the progress of the aspiration of the mist from the chamber.

### TECHNICAL FIELD

Embodiments generally relate to a spacer device for an inhaler and a method of manufacture thereof.

### BACKGROUND

The recommended treatment for asthma is inhaled administration of corticosteroids (preventive) and β2-agonists (relief of symptoms). Inhaler devices such as pressurized metered-dose inhaler (MDI) are recommended for all ages, especially for inhaling corticosteroids which has less side effects in upper airways (such as oral candidiasis). Further, such MDI attached to spacers are generally recommended for children under the age of 6 years, as per guidelines from the Global Initiative for Asthma. The spacer overcomes difficulties in coordinating the timing of inhalation and slows down the speed of delivery to reduce the deposition in the throat.

However, there are two major issues with childhood asthma management, namely compliance (especially with preventive medications) and technique (especially in young children less than three years of age). Regarding technique, the child usually does not know how to execute a proper breathing technique for inhaling the medication from the spacer. The caregiver is also unable to tell whether the child has effectively and efficiently inhaled the medication. Conventionally, the caregiver and the child merely count the number of breaths as a rough indication of whether the child has inhaled sufficient medication. But such counting method is inaccurate and insufficient inhalation of the medication can occur. Accordingly, suboptimal adherence with preventive medication can happen and may be associated with morbidity and healthcare costs.

### SUMMARY

According to various embodiments, there is provided a spacer device for an inhaler. The device may include a hollow tube. The device may also include a piston member configured to be inserted into the hollow tube such that a space between a first end of the hollow tube and the piston member may define a chamber for holding a mist or a medicated mist or a medication or an aerosolized medicine released from the inhaler. The piston may be slidable along the hollow tube towards the first end of the hollow tube as the mist or the medicated mist or the medication or the aerosolized medicine is aspirated from the chamber. The hollow tube and the piston member may cooperate to provide a visual indication of the progress of the aspiration of the mist or the medicated mist or the medication or the aerosolized medicine from the chamber.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1 shows a schematic diagram of a spacer device for an inhaler or a canister of an inhaler according to various embodiments;

FIG. 2 shows an enlarged view of a piston member of the spacer device of FIG. 1 in engagement with an inner wall of a hollow tube of the spacer device of FIG. 1 according to various embodiments;

FIG. 3 shows a spacer device for an inhaler or a canister of an inhaler according to various embodiments;

FIG. 4 shows a disassembled view of the spacer device of FIG. 3 according to various embodiments;

FIG. 5 shows a photograph of a user using the spacer device of FIG. 3 with an inhaler mask coupled to the spacer device according to various embodiments; and

FIG. 6A and FIG. 6B illustrate how a canister may be stored within the spacer device of FIG. 3 according to various embodiments.

### DETAILED DESCRIPTION

Embodiments described below in context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments of a spacer device for an inhaler or a canister of an inhaler have been provided to address at least some of the issues identified earlier. According to various embodiments, the spacer device may be referred to as a spacer, an asthma spacer, a holding chamber, etc.

Various embodiments of the spacer device seek to improve compliance via the inclusion of a feature to provide visual feedback or visual indication on the volume or amount of the medication inhaled.

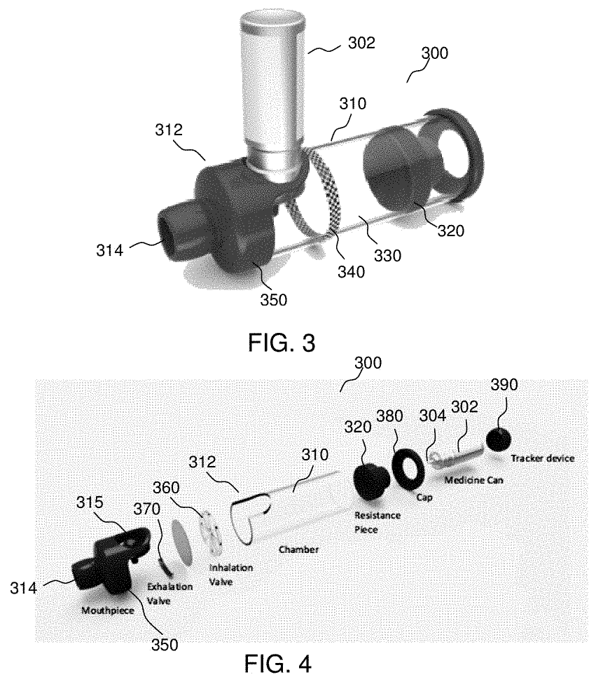

Various embodiments of the spacer device also seek to improve proper administration via the inclusion of an indicator (for example, in the form of a piston member) that provide visual feedback or visual indication on the volume or amount of the medication inhaled (for example, see FIG. 3) as well as allow the caregiver to monitor the inhalation process. According to various embodiments, upon inhalation, the indicator may move towards a “finish line” (or a marker) showing a visual cue as the medication is being depleted from the spacer while keeping the density of the medication equal as each breath is taken. According to various embodiments, the indicator may be secured by a resistance piece and may only shift when a proper deep breath is drawn, encouraging proper technique to be used.

According to various embodiments, the spacer device may be a mechanical device which provides visual feedback or visual indication of the aerosol or mist volume absorbed by the user during usage of the spacer device with the inhaler or the canister of the inhaler.

Typically, a conventional asthma spacer may be coupled with an inhaler. The inhaler may be coupled to one end of the conventional asthma spacer and a mouth-mask may be coupled to another end of the conventional asthma spacer. The inhaler may fill a spacer chamber of the conventional asthma spacer with a drug aerosol or mist when activated for a puff. Ideally, the user is typically required to inhale most of the drug volume inside the conventional asthma spacer for compliance. To achieve compliance, instructions are typically provided to users, e.g. to take 3 deep breaths through the asthma spacer. However, this is difficult to achieve, even more with children (for example, the user may question what is a deep breath) and no feedback is provided.

According to various embodiments, the spacer device may be provided with a visual indicator to show the user clearly how much of the volume has been aspirated and when the aspiration has reached close to full absorption of the drug. According to various embodiments, the visual indicator may work similar to the principle of a piston.

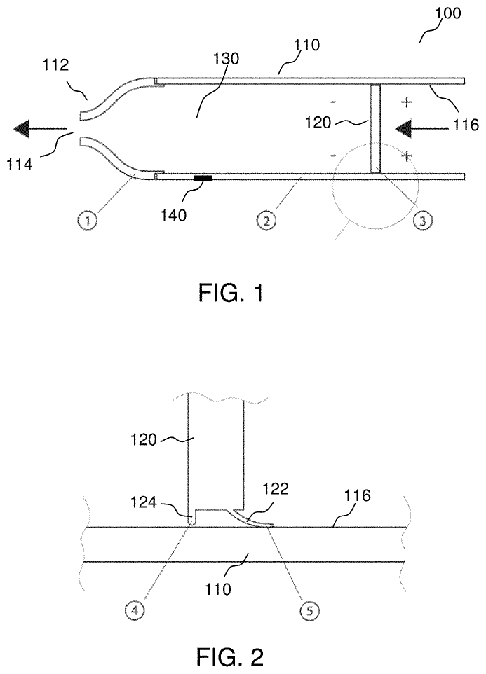

FIG. 1 shows a schematic diagram of a spacer device 100 for an inhaler or a canister of an inhaler according to various embodiments. As shown, the spacer device 100 may include a hollow tube 110 having a first end 112. The first end 112 may be configured for introducing a mist or a medicated mist or a medication or an aerosolized medicine released from the inhaler into the hollow tube 110. The first end 112 may be further configured for the mist or the medicated mist or the medication or the aerosolized medicine to be aspirated from within the hollow tube 110. According to various embodiments, the first end 112 may include one or more ports 114 which may be configured for introducing the mist or the medicated mist or the medication or the aerosolized medicine released from the inhaler and/or for the mist or the medicated mist or the medication or the aerosolized medicine to be aspirated from the hollow tube 110.

FIG. 1 further shows that the spacer device 100 may include a piston member 120 inserted into the hollow tube 110. According to various embodiments, the hollow tube 110 may be a cylindrical hollow tube. Accordingly, the piston member 120 may include a cylindrical body.

According to various embodiments, the piston member 120 may be configured to sealingly engage against an inner wall 116 of the hollow tube 110 such that a space between the first end 112 of the hollow tube 110 and the piston member 120 may define a chamber 130 for holding the mist or the medicated mist or the medication or the aerosolized medicine released from the inhaler. Further, the piston member 120 may be configured to be slidable along the hollow tube 110 towards the first end 112 as the mist or the medicated mist or the medication or the aerosolized medicine is aspirated from the chamber 130.

According to various embodiments, the user may inhale from a volume (or the spacer chamber 130) which is sealed to the side of the user mouthpiece and inhaler (or the first end 112 of the hollow tube 110) while the other side of the volume is closed by the sliding indicator (or the piston member 120). The piston member 120 may include a feature which seals the piston member 120 to the wall of the hollow tube 110 of the spacer device 100. The spacer chamber 130 may contain the mist or the drug aerosol or the medicated mist or the medication or the aerosolized medicine introduced from the inhaler or the canister of the inhaler.

According to various embodiments, when an air volume is aspirated and therefore is removed from the chamber 130, the pressure in the chamber 130 may initially drop and the piston member 120 may slide due to the higher ambient pressure outside the chamber 130. The ambient pressure may push the piston member 120 in the direction of the internal volume of the chamber 130 which has the lower pressure. Accordingly, the piston member 120 may move or slide towards the first end 112 of the hollow tube 110. The piston member 120 may stop sliding when there is again a balance of pressure, i.e. when the pressure inside the chamber 130 is similar to the ambient pressure. At this position, the piston member 120 may have moved or slided corresponding to the volume which has been aspirated by the user from the chamber 130. When more volume is aspirated again, the piston member 120 may move or slide further. According to various embodiments, the user may repeatedly inhale from the chamber 130 until the piston member 120 hits a stop when most of the aerosol volume or the desired volume has been absorbed by the user. The desired volume aspirated may correspond to the desired dosage of the mist or the medicated mist or the medication or the aerosolized medicine to be inhaled by the user. According to various embodiments, the stop may be a physical barrier for preventing further movement of the piston member 120. According to various embodiments, the stop may be the first end 112 of the hollow tube 110. According to various other embodiments, the stop may also be a protrusion which extends inward from an inner surface of the hollow tube 110. The protrusion may function as a physical limit to prevent the piston member 120 from moving further towards the first end 112 of the hollow tube 110. Accordingly, the protrusion may be a projection, or a ledge, or a flange, or a step, or a ridge, or a rib, or a bulge. According to various embodiments, the stop may be any suitable feature which ends the travel for the piston member 120 in a predetermined position that ensures low residual volume.

According to various embodiments, the hollow tube 110 and the piston member 120 may cooperate to provide a visual indication of the progress of the aspiration of the mist or the medicated mist or the medication or the aerosolized medicine from the chamber 130 (or the progress of the inhalation of the mist or the medicated mist or the medication or the aerosolized medicine from the chamber 130 by the user). According to various embodiments, the hollow tube 110 may include at least a see-through portion while the piston member 120 may be opaque and/or coloured. The see-through portion may be a transparent or a translucent portion of the hollow tube 110. Accordingly, the transparent or translucent portion of the hollow tube 110 may allow the piston member 120 to be visible from outside of the hollow tube 110 for allowing one to observe the movement of the piston member 120 from outside such that the hollow tube 110 and the piston member 120 may provide the visual indication of the progress of the aspiration of the mist or the medicated mist or the medication or the aerosolized medicine from the chamber 130 as the piston member 120 (being visible through the transparent or translucent portion of the hollow tube 110) moves or slides along the hollow tube 110 due to the aspiration of the mist or the medicated mist or the medication or the aerosolized medicine from the chamber 130.

According to various embodiments, the entire hollow tube 110 may be transparent or translucent such that the piston member 120 may be visible from the outside of the hollow tube 110. According to various other embodiments, the transparent or translucent portion of the hollow tube 110 may be a transparent or translucent strip extending lengthwise along the hollow tube 110. In one embodiment, the transparent or translucent portion may extend the entire length of the hollow tube 110. In another embodiment, the transparent or translucent portion may extend only a predetermined portion of the length of the hollow tube 110. According to various embodiments, the spacer device 100 may further include a marker 140 on the hollow tube 110. Accordingly, the hollow tube 110 having the transparent or translucent portion and the markers may cooperate with the piston member 120 to provide visual indication of a progress of aspiration of the mist or the medicated mist or the medication or the aerosolized medicine from the chamber 130. For example, the marker 140 may be configured to demarcate the final position of the piston member 120 when a desired volume has been aspirated from the chamber 130. The desired volume aspirated may correspond to the desired dosage to be inhaled by the user. Accordingly, relative positions between the piston member 120 (as seen through the transparent or translucent portion of the hollow tube 110) and the marker 140 on the hollow tube 110 may provide a visual indication of the progress of aspiration of the mist or the medicated mist or the medication or the aerosolized medicine from the chamber 130. Further, when the piston member 120 reaches the marker 140, the piston member 120 and the marker 140 on the hollow tube 110 may provide a visual indication of the completion of the aspiration of the desired volume of mist or the medicated mist or the medication or the aerosolized medicine from the chamber 130. According to various other embodiments, the spacer device 100 may include one or more markers 140 on the hollow tube 110. Each of the one or more markers 140 may demarcate a position of the piston member 120 corresponding to a calibrated cumulative volume that has been aspirated from the chamber 130. Accordingly, the piston member 120 reaching any one of the one or more markers 140 on the hollow tube 110 may be a visual indication of the corresponding cumulative volume of the mist or the medicated mist or the medication or the aerosolized medicine (associated with the respective marker 140) being aspirated from the chamber 130. According to various embodiments, the spacer device 100 may further include a stopper on an inner surface of the hollow tube 110 at the position of the marker 140. The stopper maybe configured to act a physical barrier to stop the piston member 120 from moving pass the marker 140 towards the first end 112 of the hollow tube 110.

According to various embodiments, the piston member 120 may be configured to be sealed to the hollow tube 110 in a way that provides as little friction as possible to ensure that the effort to aspirate and move the piston member 120 may be low or sufficient to be considered a proper breath. Also the piston member 120 may be configured to be guided so as to avoid any tilting which may compromise sealing or cause jamming, or both.

FIG. 2 shows an enlarged view of the piston member 120 in engagement with the inner wall 116 of the hollow tube 110 according to various embodiments. As shown, the piston member 120 may include a skirting 122. Accordingly, the sealing between the piston member 120 and the hollow tube 110 may be achieved by the skirting 122 on the edge of the piston member 120 or around a perimeter of the piston member 120. According to various embodiments, the skirting 122 may be configured to be deflected to uninterruptedly seal the piston member 120 against the inner wall 116 of the hollow tube 110 along the perimeter of the piston member 120 when the piston member 120 is inserted into the hollow tube. According to various embodiments, the skirting 122 may have, in its original shape, (theoretical) interference with the chamber wall or the inner wall 116 of the hollow tube 110. Accordingly, the skirting 122 of the piston member 120 may be dimensioned for interference fit with the hollow tube 110. When inserted into the hollow tube 110, the skirting 122 may be deflected inwards such that the skirting 122 may have an uninterrupted circular line contact (or all-round contact) with the inner wall 116 of the hollow tube 110. According to various embodiments, the dimension of the required deflection of the skirting 122 may depend mostly on manufacturing tolerances, such that with natural imprecision of the parts there may never be a gap forming between the piston member 120 and the chamber wall which may compromise the seal, independent of the position and the rotation of the piston member 120. Accordingly, the sealing between the piston member 120 and the inner wall 116 of the hollow tube 110 may be free of any gaps. According to various embodiments, the deflection of the skirting 122 may be kept to a minimum in order to reduce friction. According to various embodiments, the skirting 122 may also be very flexible, so that there may only be a minimum force exerted on the chamber wall for a given deflection, as this force may contribute to friction and may make aspiration more difficult.

According to various embodiments, the skirting 122 may be configured to contact the inner wall 116 of the hollow tube 110 such that a force acting on the piston member 120 along a direction towards the first end 112 of the hollow tube 110 caused by the drop in pressure when a predetermined volume is aspirated from the chamber 130 may be greater than a friction caused by the contact between the skirting 122 and the inner wall 116 of the hollow tube 110 so as to move or slide the piston member 120 along the hollow tube 110 in the direction towards the first end 112 of the hollow tube 110 upon the aspiration of the predetermined volume from the chamber 130. According to various embodiments, the predetermined volume to be aspirated may correspond to a proper breath or a deep breath or a breath taken with the correct technique as required when using the spacer device 100. Accordingly, if the user did not use the appropriate technique for inhaling from the chamber 130, the user may not inhale the predetermined volume from the chamber 130. Hence, the piston member 120 may not move or slide as the force generated may not be sufficient to overcome the friction between the skirting 122 and the inner wall 116 of the hollow tube 110.

According to various embodiments, the piston member 120 may include a guide 124 configured to prevent jamming or tilting (which may cause leakage). The guide 124 may be configured to achieve alignment of the axis of the piston member 120 with a centre line of the chamber 130. Accordingly, the guide 124 of the piston member 120 may be configured to cooperate with the skirting 122 to align an axis of the piston member 120 with a longitudinal axis of the hollow tube 110 when the piston member 120 is inserted into the hollow tube 110. Hence, the guide 124 may work together with the skirting 122 such that the piston member 120 may not tilt so as to ensure sealing between the piston member 120 and the hollow tube 110. Thus, the piston member 120 may be configured such that the guide 124 may be disposed relative to the skirting 122 in a predetermined disposition so as to ensure alignment of the skirting 122 with the longitudinal axis of the hollow tube 110 during movement of the piston member 120. According to various embodiments, the guide 124 and the skirting 122 may be separate components of the piston member 120 and may be rigidly connected or mated or joined together to form the piston member 120. According to various other embodiments, the piston member 12 may be integrally formed or moulded or three-dimensionally printed as a single body with the guide 124 and the skirting 122.

According to various embodiments, the guide 124 of the piston member 120 may be dimensioned for sliding clearance fit with the hollow tube 110. According to various embodiments, the guide 124 may be a sliding guide in ring form, touching the chamber wall, or the guide 124 may include protrusions in several points around the circumference of the piston member 120. Accordingly, the guide 124 may include one or more protrusions extending radially from the piston member 120. In one embodiment, the guide 124 may also be a flange projecting radially from the piston member 120. According to various embodiments, the guide 124 may be attached to the piston member 120 in order to align it relative to the inner chamber wall (or the inner wall 116 of the hollow tube 110). According to various embodiments, the guide 124 may be set apart from the skirting 122 (or configured to have a distance between the guide 124 and the skirting 122). In one embodiment, when the piston member 120 is in the hollow tube 110, the guide 124 and the skirting 122 may be configured such that the guide 124 may be directed towards the first end 112 of the hollow tube 110 (or in front of the piston member 120 in a direction towards the first end 112 of the hollow tube 110). In another embodiment, when the piston member 120 is in the hollow tube 110, the guide 124 and the skirting 122 may be configured such that the skirting 122 may be directed towards the first end 112 of the hollow tube 110 (or in front of the piston member 120 in a direction towards the first end 112 of the hollow tube 110).

FIG. 3 shows a spacer device 300 for an inhaler or a canister 302 of an inhaler according to various embodiments. FIG. 4 shows a disassembled view of the spacer device 300 of FIG. 3 according to various embodiments. As shown, the spacer device 300 may include a hollow tube 310 having a first end 312. The first end 312 may be configured for introducing a mist or a medicated mist or a medication or an aerosolized medicine released from the canister 302 into the hollow tube 310. The first end 312 may be further configured for the mist or the medicated mist or the medication or the aerosolized medicine to be aspirated from within the hollow tube 310. According to various embodiments, the first end 312 may be configured to include one or more ports 314, 315 which may be configured for introducing the mist or the medicated mist or the medication or the aerosolized medicine released from the canister 302 and/or for the mist or the medicated mist or the medication or the aerosolized medicine to be aspirated from the hollow tube 310.

FIG. 3 and FIG. 4 further shows that the spacer device 300 may include a piston member 320 configured to be inserted into the hollow tube 310. As shown, the hollow tube 310 may be a cylindrical hollow tube and the piston member 320 may include a cylindrical body inserted in the cylindrical hollow tube.

According to various embodiments, the piston member 320 may be similar to the piston member 120 of FIG. 1 and FIG. 2. Accordingly, the piston member 320 may be configured to sealingly engage against an inner wall 316 of the hollow tube 310 such that a space between the first end 312 of the hollow tube 310 and the piston member 320 may define a chamber 330 for holding the mist or the medicated mist or the medication or the aerosolized medicine released from the canister 302. Further, the piston member 320 may be configured to be slidable along the hollow tube 310 towards the first end 312 as the mist or the medicated mist or the medication or the aerosolized medicine is aspirated from the chamber.

According to various embodiments, the hollow tube 310 and the piston member 320 may be configured, similar to the various configuration of the hollow tube 110 and the piston member 120 as described herein, such that the hollow tube 310 and the piston member 320 may cooperate to provide a visual indication of the progress of the aspiration of the mist or the medicated mist or the medication or the aerosolized medicine from the chamber 330.

As shown in FIG. 3 and FIG. 4, the entire hollow tube 310 may be transparent or translucent. Further, the hollow tube 310 may be configured to include a marker 340 (in the form of a “finish line”) on the hollow tube 310. The marker 340 on the hollow tube 310 may demarcate the final position of the piston member 320 when a desired volume has been aspirated from the chamber 330. The desired volume aspirated may correspond to the desired dosage to be inhaled by the user. Accordingly, relative positions between the piston member 320 (as seen through the transparent or translucent hollow tube 310) and the marker 340 on the hollow tube 310 may provide a visual indication of the progress of aspiration of the mist or the medicated mist or the medication or the aerosolized medicine from the chamber 330. Further, when the piston member 320 reaches the marker 340, the piston member 320 and the marker 340 on the hollow tube 310 may provide a visual indication of the completion of the aspiration of the desired volume of the mist or the medicated mist or the medication or the aerosolized medicine from the chamber 330. Hence, the transparent hollow tube 310 having the marker 340 may cooperate with the piston member 320 to provide a visual indication of the progress and/or the completion of the desired aspiration when the piston member 320 being visible through the transparent hollow tube 310 moves/slides relative to the marker 340 and/or reaches the marker 340. Accordingly, the visual indication provided by the transparent hollow tube 310 having the marker 340 and the piston member 320 may allow the user to observe the movement of the piston member 320 as the user draws in the breath and to aim to empty the chamber 330 up to the marker 340 (or the “finishing” line).

According to various embodiments, the piston member 320 may, similar to the piston member 120 of FIG. 1 and FIG. 2, include a skirting and a guide. The skirting and the guide may be configured in a similar manner according to the various configurations of the skirting 122 and the guide 124 as described herein.

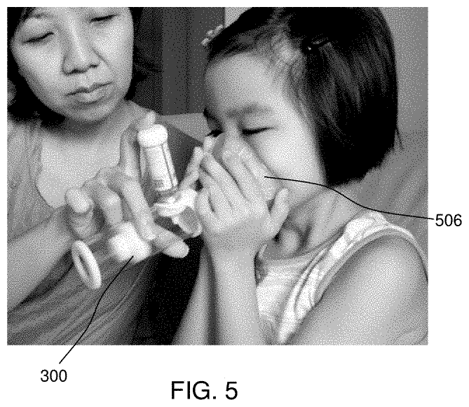

Further, as shown in FIG. 3 and FIG. 4, the spacer device 300 may include a connector 350 configured to be coupled to the first end 312 of the hollow tube 310. The connector 350 may include a first port 315 configured for coupling with the canister 302. The first port 315 may be configured to direct the mist or the medicated mist or the medication or the aerosolized medicine released from the canister 302 through the first port 315 into the hollow tube. According to various embodiments, the first port 315 may be configured to couple with a nozzle 304 of the canister 302. As shown, the connector 350 may include a second port 314 configured for aspiration of the mist or the medicated mist or the medication or the aerosolized medicine from the chamber 330. The second port 314 may be in the form of a conduit and may be configured for coupling with an inhaler mask 506 (see FIG. 5). FIG. 5 shows a photograph of a user using the spacer device 300 with the inhaler mask 506 coupled to the second port 314.

In FIG. 4, the spacer device 300 is shown to include a first one-way valve 360. When assembled, the first one-way valve 360 may be disposed between the second port 314 of the connector 350 and the hollow tube 310. The first one-way valve may be configured such that, during aspiration of the mist or the medicated mist or the medication or the aerosolized medicine from the chamber 330 via the second port 314, the mist or the medicated mist or the medication or the aerosolized medicine may flow in the direction from the chamber 330 through the first one-way valve 360 before reaching and exiting the second port 314 of the connector 350. Accordingly, the one-way valve 360 may prevent air flow in the opposite direction, i.e prevent exhaled air from the user to flow through the first one-way valve 360 into the chamber 330. According to various embodiments, the spacer device 300 may include a second one-way valve 370. The second one-way valve may be disposed in a portion of a body of the connector 350 such that inbound exhaled air from the user through the second port 314 of the connector 350 may be directed to exit the connector 350 via the second one-way valve 370. Accordingly, the second one-way valve 370 may be configured to direct external airflow entering the second port 314 of the connector 350 to exit the connector 350 through the second one-way valve 370 during exhalation by the user.

Referring back to FIG. 3 and FIG. 4, the spacer device 300 may further include a cap 380 configured to be coupled to a second end 318 of the hollow tube 310. The cap 380 may function as a physical stopper for abutting the piston member 320 at the start position before usage of the spacer device 300. As shown, the cap 380 may include an aperture in its centre and the piston member 320 may include a protruding portion configured to be fitted into the aperture.

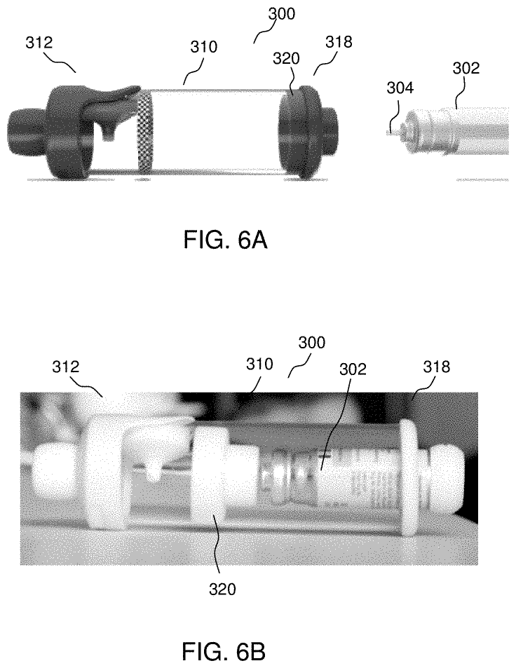

FIG. 6A and FIG. 6B illustrate how the canister 302 may be stored within the spacer device 300 according to various embodiments. As shown, the piston member 320 may be configured for coupling with the canister 302 such that the canister 302 may be held by the piston member 320. According to various embodiments, the piston member 320 may be configured to couple with the nozzle 304 of the canister 302. Accordingly, the piston member 320 (coupled to the canister 302) may be pushed towards the first end 312 of the hollow tube 310 as the canister 302 is inserted into the hollow tube 310 for storage. Accordingly, the spacer device 300 may be configured to house the canister 302 during storage to save space and improve portability. Hence, the spacer device 300 may provide a storage space for the canister and thus occupy less storage form factor.

Subsequently, when the canister 302 is being removed from the hollow tube 310, the piston member 320 being coupled to the canister 302 may be pulled towards the second end 318 of the hollow tube 310. As the piston member 320 reaches the second end 318 of the hollow tube 310, the cap 380 may stop the piston member 320 from being pulled out of the hollow tube 310 and, at the same time, the canister 302 may be decoupled from the piston member 320. Accordingly, upon removal of the canister 302 from the spacer device 300, the spacer device 300 may be prepared, set and ready for usage.

Referring back to FIG. 4, the spacer device 300 may further include a tracker 390 (or an electronic monitoring module). The tracker 390 may be configured to be integrated with the piston member 320. The tracker 390 may be configured to include one or more sensors for detecting complete inhalation only when the piston member 320 (or the visual indicator) reaches the marker 340 (or the “finishing line”). The tracker 390 may be configured for electronic monitoring of the inhalation based on the movement of the piston member 320.

According to various embodiments, there is provided a spacer device for an inhaler. The device may include a hollow tube. The device may also include a piston member configured to be inserted into the hollow tube such that a space between a first end of the hollow tube and the piston member may define a chamber for holding a mist or a medicated mist or a medication or an aerosolized medicine released from the inhaler. The piston may be slidable along the hollow tube towards the first end of the hollow tube as the mist or the medicated mist or the medication or the aerosolized medicine is aspirated from the chamber. The hollow tube and the piston member may cooperate to provide a visual indication of the progress of the aspiration of the mist or the medicated mist or the medication or the aerosolized medicine from the chamber

According to various embodiments, the hollow tube may include at least a transparent or translucent portion. The transparent or translucent portion of the hollow tube may extend lengthwise along the hollow tube.

According to various embodiments, the spacer device may further include a marker on the hollow tube. The hollow tube may have the transparent or translucent portion. The marker may cooperate with the piston member to provide a visual indication of a completion of a desired aspiration when a portion of the piston member visible through the transparent or translucent portion of the hollow tube reaches the marker.

According to various embodiments, the piston member may include a skirting around a perimeter of the piston member. The skirting may be configured to be deflected to uninterruptedly seal the piston member against an inner wall of the hollow tube along the perimeter of the piston member when the piston member is inserted into the hollow tube.

According to various embodiments, the skirting may be configured to contact the inner wall of the hollow tube such that a force acting on the piston member towards the first end caused by an aspiration of a predetermined volume of the mist or the medicated mist or the medication or the aerosolized medicine from the chamber may be greater than a friction caused by the contact between the skirting and the inner wall of the hollow tube so as to slide the piston member along the hollow tube towards the first end upon the aspiration of the predetermined volume of the mist or the medicated mist or the medication or the aerosolized medicine from the chamber.

According to various embodiments, the piston member may further include a guide configured to cooperate with the skirting to align an axis of the piston member with a longitudinal axis of the hollow tube when the piston member is inserted into the hollow tube. The guide may include one or more protrusions extending radially from the piston member. The guide may include a flange projecting from the piston member.

According to various embodiments, the spacer device may further include a connector configured to be coupled to the first end of the hollow tube.

According to various embodiments, the connector may include a first port which may be configured for coupling with the inhaler and which may be configured to direct the mist or the medicated mist or the medication or the aerosolized medicine released from the inhaler through the first port into the hollow tube.

According to various embodiments, the connector may include a second port which is configured for aspiration of the mist or the medicated mist or the medication or the aerosolized medicine from the hollow tube. The second port may be configured for coupling with an inhaler mask.

According to various embodiments, the spacer device may further include a first one-way valve disposed between the second port of the connector and the hollow tube. The first one-way valve may be configured for the aspiration of the mist or the medicated mist or the medication or the aerosolized medicine from the chamber, through the first one-way valve, and out from the second port of the connector.

According to various embodiments, the spacer device may further include a second one-way valve disposed at the connector. The second one-way valve may be configured to direct external airflow entering the second port of the connector to exit the connector through the second one-way valve during exhalation by the user.

According to various embodiments, The piston member may be configured for coupling with the inhaler for storing the inhaler within the hollow tube.

According to various embodiments, there is provided a method of manufacturing a spacer device for an inhaler. The method may include providing a hollow tube. The method may further include providing a piston member configured to be inserted into the hollow tube such that a space between a first end of the hollow tube and the piston member may define a chamber for holding a mist or a medicated mist or a medication or an aerosolized medicine released from the inhaler. The piston member may be slidable along the hollow tube towards the first end of the hollow tube as the mist or the medicated mist or the medication or the aerosolized medicine is aspirated from the chamber. The hollow tube and the piston member may cooperate to provide a visual indication of the progress of the aspiration of the mist or the medicated mist or the medication or the aerosolized medicine from the chamber.

According to various embodiments, the method may further include configuring the spacer device according to various embodiments of the spacer device as described herein.

Various embodiments have provided a spacer device configured to improve compliance and breathing technique. According to various embodiments, the spacer device may incorporate a visual indicator (may be in the form of a piston member) to monitor the overall inhalation process. On inhalation, the indicator may move towards a “finish line” (or a marker) showing a visual cue as the medication is being depleted from the spacer device while keeping the density of the medication equal as each breath is taken. The indicator which may be secured by a resistance piece may only shift when a proper deep breath is drawn, encouraging proper technique to be used. In contrast, the spacer device according to various embodiments is unlike conventional spacers which have no clear indication as to whether the user/child is breathing in properly. As the indicator of the spacer device according to various embodiments reaches the “finish line” on the spacer device, this may reveal that the user/child has successfully inhaled the required dosage of the medication. Accordingly, various embodiments greatly reduce the need to inhale multiple deep breaths, as conventionally suggested by doctors (conventional method requires children to take 8 to 10 breaths to ensure adequate inhalation which results in a tedious and inefficient process).

Various embodiments have also provided a spacer device configured for ease of storage. Various embodiments may incorporate a feature to house the inhaler or canister during storage to save space and to improve portability. Upon removal the inhaler or the canister from the spacer device, the spacer device may be set and ready for use.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. A spacer device for an inhaler, the device comprising:
a hollow tube; and
a piston member configured to be inserted into the hollow tube such that a space between a first end of the hollow tube and the piston member defines a chamber for holding a mist released from the inhaler, wherein the piston member is slidable along the hollow tube towards the first end of the hollow tube by a force resulting from a pressure differential between an ambient pressure acting on the piston member from an open second end of the hollow tube opposite the first end and a chamber pressure lower than the ambient pressure as the mist is aspirated from the chamber,
wherein the hollow tube and the piston member cooperate to provide a visual indication of the progress of the aspiration of the mist from the chamber.

2. The device as claimed in claim 1, wherein the hollow tube comprises at least a transparent or translucent portion.

3. The device as claimed in claim 2, wherein the transparent or translucent portion of the hollow tube extend lengthwise along the hollow tube.

4. The device as claimed in claim 3, further comprising a marker on the hollow tube, wherein the hollow tube having the transparent or translucent portion and the marker cooperates with the piston member to provide a visual indication of a completion of a desired aspiration when a portion of the piston member visible through the transparent or translucent portion of the hollow tube reaches the marker.

5. The device as claimed in claim 1, wherein the piston member comprises a skirting around a perimeter of the piston member, and wherein the skirting is configured to be deflected to uninterruptedly seal the piston member against an inner wall of the hollow tube along the perimeter of the piston member when the piston member is inserted into the hollow tube.

6. The device as claimed in claim 5, wherein the skirting is configured to contact the inner wall of the hollow tube such that a force acting on the piston member towards the first end caused by an aspiration of a predetermined volume of the mist from the chamber is greater than a friction caused by the contact between the skirting and the inner wall of the hollow tube so as to slide the piston member along the hollow tube towards the first end upon the aspiration of the predetermined volume of the mist from the chamber.

7. The device as claimed in claim 5, wherein the piston member further comprises a guide configured to cooperate with the skirting to align an axis of the piston member with a longitudinal axis of the hollow tube when the piston member is inserted into the hollow tube.

8. The device as claimed in claim 7, wherein the guide comprises one or more protrusions extending radially from the piston member.

9. The device as claimed in claim 7, wherein the guide comprises a flange projecting from the piston member.

10. The device as claimed in claim 1, further comprising a connector configured to be coupled to the first end of the hollow tube.

11. The device as claimed in claim 10, wherein the connector comprises a first port which is configured for coupling with the inhaler and which is configured to direct the mist released from the inhaler through the first port into the hollow tube.

12. The device as claimed in claim 10 or 11, wherein the connector comprises a second port which is configured for aspiration of the mist from the hollow tube.

13. The device as claimed in claim 12, wherein the second port is configured for coupling with an inhaler mask.

14. The device as claimed in claim 12, further comprising a first one-way valve disposed between the second port of the connector and the hollow tube, wherein the first one-way valve is configured for the aspiration of the mist from the chamber, through the first one-way valve, and out from the second port of the connector.

15. The device as claimed in claim 14, further comprising a second one-way valve disposed at the connector, the second one-way valve configured to direct external airflow entering the second port of the connector to exit the connector through the second one-way valve during exhalation by the user.

16. The device as claimed in claim 1, wherein the piston member is configured for coupling with the inhaler for storing the inhaler within the hollow tube.

17. A method of manufacturing a spacer device for an inhaler, the method comprising:
providing a hollow tube; and
providing a piston member configured to be inserted into the hollow tube such that a space between a first end of the hollow tube and the piston member defines a chamber for holding a mist released from the inhaler, wherein the piston member is slidable along the hollow tube towards the first end of the hollow tube by a force resulting from a pressure differential between an ambient pressure acting on the piston member from an open second end of the hollow tube opposite the first end and a chamber pressure lower than the ambient pressure as the mist is aspirated from the chamber,
wherein the hollow tube and the piston member cooperate to provide a visual indication of the progress of the aspiration of the mist from the chamber.

18. The method as claimed in claim 17, further comprising configuring the spacer device according to claim 1.
